(19) United States
(12) Patent Application Publication
(10) Pub. No.: US 2005/0065336 A1
(43) Pub. Date: Mar. 24, 2005

(54) Method for separating xylose from lignocelluloses rich in xylan, in particular wood

(76) Inventor: Ties Karstens, Boetzingen (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/496,853

(22) PCT Filed: Nov. 27, 2002
(86) PCT No.: PCT/EP02/13377

(30) Foreign Application Priority Data
Nov. 27, 2001 (DE) 101 58 120.3

(51) Int. Cl. C07H 1/00

(52) U.S. Cl. 536/124

(57) ABSTRACT

The invention is directed to a method for separating xylose from lignocelluloses rich in xylan, particularly wood, and for obtaining pulp, characterized by the following steps: (1) pretreating wood chips through mechanical destruction of the original structure; (2) impregnating the obtained wood mass with diluted mineral acid; (3) carrying out prehydrolysis of the obtained wood mass modified by the process under the influence of steam at an elevated temperature to hydrolyze the obtained hemicelluloses; and (4) removing the hemicelluloses from the residual pulp by washing, filtering and/or centrifuging while obtaining an aqueous solution rich in xylose. The combination of method steps according to the invention makes it possible to achieve high α-cellulose contents with very low proportions of xylose, that is, highly pure chemical pulp qualities, while at the same time enabling a virtually quantitative separation of the valuable xylose.

### CROSS-RFERENCE TO RELATED APPLICATIONS

This application claims priority of International Application No. PCT/EP02/13377, filed Nov. 27, 2002 and German Application No. 101 58 120.3, filed Nov. 27, 2001, the complete disclosures of which are hereby incorporated by reference.

### BACKGROUND OF THE INVENTION

a) Field of the Invention

The invention is directed to a method for separating xylose from lignocelluloses rich in xylan, particularly wood, and for obtaining pulp.

b) Description of the Related Art

For commercial utilization of wood, this natural composite comprising the backbone substance in the form of cellulose chains twisted into long, strong fibrils and the hydrophobic lignin acting as a protective sheathing must be destroyed in order to separate these constituents from one another subsequently in pulping or defibration, as it is called. Hemicelluloses (polyoses) are another essential constituent of wood. Usually, the aim is to obtain more or less pure cellulose; the other constituents, lignin and hemicelluloses, usually only occur in decomposed form as secondary product

Pulps are differentiated into paper pulp which can contain up to 25% hemicellulose in addition to the main constituent, α-cellulose, and the higher-grade chemical pulp which has an a-cellulose proportion of over 90% and whose proportion of hemicellulose should not exceed a few percent. As is well known, chemical pulp is one of the most important cost factors in the production of cellulose acetate, filter tow and other highly valuable cellulose products.

Therefore, in order to produce chemical pulps of this kind from wood or comparable biomasses, in addition to removing the lignin, it is also necessary to separate the hemicelluloses as much as possible. In this connection, hemicelluloses have become increasingly important because of their useful possibilities.

Numerous methods have been developed in the prior art for chemical decomposition and removal of xylose from wood. A classic method for the removal of xylose makes use of an acid prehydrolysis which is carried out in practice either with diluted sulfuric acid in a concentration of 0.1 to 0.5% and at a temperature of 90° C. to 140° C. or in pure water at a temperature of 165° C. to 175° C. (H. Sixta, G. Schild and Th. Baldinger: Paper [Das Papier], volume 9, 1992, pages 527 ff.). Typically about 70% of the xylose is removed during prehydrolysis. Of the remaining 30%, 40 to 50%, i.e., 12% to 15% absolute, of the xylose is removed during cooking. Accordingly, in all, 82% to 85% of the xylose present is removed from the wood.

The hydrolysis is carried out as water hydrolysis, i.e., with bath ratios of 3 to 4:1. The heating-up time is at least 60 minutes and is usually as much as 90 minutes. The hydrolysate is usually disposed of thermally along with the cooking liquor. There has been no shortage of attempts to reduce the bath ratio to the pure steam prehydrolysis (bath ratio of 1:1 to 1:5.1) in order to reduce the high energy costs incurred in evaporating the prehydrolysate. Unfortunately, however, this technologically very simple process has a very negative effect on pulp quality. Tests have shown that steam prehydrolysis is a definite cause of higher kappa numbers, poorer bleachability, lower alkali resistance, and reactivity of pulps.

Further, prehydrolysis is limited by the occurrence of secondary reactions which are difficult to control. The most important secondary reaction, the dehydration of pentoses to form furfural, is the starting point for unwanted intermolecular and intramolecular condensation reactions. This results in resinous compounds which separate from the aqueous phase as the reaction continues and which can be deposited on all surfaces present (gumming and clogging in regular operation).

In accordance with the article, cited above, by H. Sixta, G. Schild and Th. Baldinger and the prehydrolysis described therein at temperatures of 164° C., 170° C., 178° C. and 185° C., the highest xylose concentrations that can be achieved after 100 minutes are listed in the following Table 1. More furfural than xylose is formed at all reaction temperatures with the exception of 164° C. The furfural concentration reaches a saturation value of 20 to 23 g/l. Reaction conditions under which the unwanted further reactions of furfural to condensation products can still be controlled to a reasonable extent for technical processes are at the maximum prehydrolysis times shown in Table 2.

TABLE 1

| | Duration of hydrolysis |
|---|---|
| | until reaching the highest |
| Hydrolysis | xylose concentration of |
| temperature | 14-15 g/l |
| [° C.] | [min] |
| 164 | 180 |
| 170 | 140 |
| 178 | 70 |
| 185 | 20 |

TABLE 2

| | Maximum |
|---|---|
| Hydrolysis | controllable |
| temperature | hydrolysis time |
| [° C.] | [min] |
| 165 | 160 |
| 171 | 90 |
| 179 | 60 |
| 186 | 30 |

The P-factor for these temperatures and times is approximately 700±5. The P-factor model is used to calculate the degree of prehydrolysis over the time-temperature profile. In so doing, temperature and time are combined in a variable, the P-factor. To this extent, the P-factor is to be considered as analogous to the severity factor introduced in the steam explosion of wood. According to the article by H. Sixta, G. Schild and Th. Baldinger, cited above, the viscosity decreases by about 20% at this P-factor. When the P-factor, i.e., the above temperatures and times, is maintained, the yield of unwanted secondary products is low. The glucose concentration is independent from the maximum reaction temperature to a great extent and is about 2 g/l.

Further, the known sources for xylose exhibit a range of disadvantages. Known xylose sources are, for example, the cooking liquors of the Mg- and Ca-sulfite methods. However, these cooking liquors must be subjected to an ion exchange in order to remove the lignin sulfonates, and additional chromatography processes are needed to separate the xylose from other sugar components, possibly crystallizations.

As is well known, hardwood paper pulps or kraft pulps are subjected to an alkaline treatment (8-12% NaOH) at 60° C. to 80° C. in order to change the xylan to the liquid phase. This xylan must then be hydrolyzed to form xylose. Xylan hydrolysate likewise results from a slurry after treating with xylanase, and xylose must be concentrated from the xylan hydrolysate by chromatography separation methods. Further, the sulfite method (cooking liquor or spent liquor) initially leads to a separation through Ca salt chromatography (ion exchanger) into a first lignin sulfonate fraction and a sugar-rich fraction. This is followed by a second separation through Na salt chromatography (ion exchanger) into another lignin sulfonate fraction and a purified sugar-rich fraction.

Consequently, there is a demand for a simple, economical method for separating hemicelluloses, particularly xylose, from wood while conserving the cellulose.

### OBJECT AND SUMMARY OF THE INVENTION

Proceeding from the prior art described above, it was the primary object of the invention to provide a method for separating xylose which works in a simple and economical manner and in which there are as few waste products as possible to be worked up. Further, a high cellulose yield is to be achieved with the method according to the invention.

This object is met, according to the invention, through a method for separating xylose from xylan-rich lignocelluloses, particularly wood, and for obtaining pulp, characterized by the following steps:
(1) pretreating wood chips through mechanical destruction of the original structure;
(2) treating the wood mass with diluted mineral acid;
(3) carrying out prehydrolysis of the obtained wood mass modified by the process under the influence of steam at an elevated temperature to hydrolyze the obtained hemicelluloses; and
(4) removing the hemicelluloses from the remaining pulp by washing, filtering and/or centrifuging while obtaining an aqueous solution rich in xylose.

The above-stated object can be met in an advantageous manner through the invention. Consequently, it contributes to a considerable improvement over the prior art. A procedure that is completely different than the conventional wood defibration method allows a wide variety of advantages over the sulfite or prehydrolysis sulfate method in the field. This is due primarily to decisive improvements in the treatment of the wood chips. These improvements are based on avoiding the disadvantages of the classic digester technology, namely, filling digesters with wood chips, long heating-up and cooking times, high bath ratios and almost insoluble problems with respect to heat exchange and mass transfer due to low diffusion rates and negligible thermodynamic potential differences. These disadvantages in turn cause a high investment requirement, i.e., large components because of long resident times.

Therefore, according to the invention, the prehydrolysis is not carried out on the wood chips themselves, but rather is carried out after pretreatment of the wood chips. This pretreatment consists in crushing and chopping wood chips initially in method step (1) by means of a suitable mechanical materials processing unit such as a pressing worm, spindle press or the like to such an extent that their original structure is destroyed. Not only is the water that is stored in the wood chips removed by this massive mechanical action, but the air is also expelled from the capillaries of the wood. In addition, the moisture is lowered appreciably, namely, by up to about 50%, by the crushing process.

Any comminuted vegetable material of many different kinds can be used such as wood, oat husks, corn stalks and grain stalks, bagasse, wheat straw, rice, straw and oat straw. With respect to wood, it is conventional to use round wood or softwood in comminuted form. Fibers which are comminuted by cutting are suitable as fibrous raw materials such as annuals. Comminuted, especially shredded, wood is preferable; the use of hardwood, particularly beech or eucalyptus, is preferred.

The mechanical action according to step (1) of the method according to the invention enables the obtained wood mass—it can no longer be called wood chips after this treatment—to soak up any type of provided liquid spontaneously and within a very short time in order to approach the original state again.

This fact can be made use of in that the wood chips which are pretreated in this way are impregnated with diluted mineral acid in step (2) of the method according to the invention. The mineral acid can be any known mineral acid. Examples include sulfuric acid, hydrochloric acid and/or nitric acid, sulfuric acid being preferred. An approximately 0.05 N to 2.5 N, particularly approximately 0.1 N to 1.5 N, diluted aqueous mineral acid solution is advantageously used.

This mineral acid solution is soaked up by the wood mass spontaneously to the extent that its liquid content after soaking is preferably between about 55% and 70%. The uniformity of distribution over the entire cross section of the wood mass can easily be detected in the change in color of the wood mass from light to dark. The distribution of the liquid is generally uniform unless the wood chips were not crushed sufficiently.

The wood mass which is pretreated in this way can be treated according to step (3) in a suitable apparatus, e.g., an inclined screw reactor, Pandia reactor or the like, by treating with steam at a temperature of about 140° C. to 180° C., particularly about 150° C. to 180° C., for approximately 5 to 40 minutes, particularly approximately 5 to 20 minutes, preferably in continuous operating mode, during which the prehydrolysate can likewise be taken off continuously. The resulting prehydrolyzed wood mass is extensively defibrated and in a very greatly softened form.

The modified wood mass obtained after carrying out the prehydrolysis according to step (3) can advisably be completely defibrated in a very simple manner and without great effort. This is accomplished, for example, by means of a refiner, a toothed disk mill, extensive chopping in a narrow pipe as a result of the reduction in steam pressure from the above-mentioned temperatures to normal pressure, and the like. The separation of fibrous materials and steam is preferably carried out in a cyclone.

In step (4), the hemicelluloses which still adhere to the fibers and are broken down into sugars can then be quantitatively separated in a washing and filtering step. A washing centrifuge, a double washing press, a traveling screen press or, very generally, a multistage washing unit is preferably used for washing.

The method has proven particularly effective for woods with a high proportion of xylan, e.g., eucalyptus pulp. Accordingly, aqueous xylose solutions of 10% to 25%, particularly about 15% to 20%, can advantageously be obtained. Through further concentration, a supersaturated solution can be obtained and the xylose can be crystallized therefrom. However, experience has shown that even 25-percent xylose solutions are economically viable for further processing.

Consequently, high α-cellulose contents and very low proportions of xylose, that is, highly pure chemical pulp qualities, can be achieved with the combination of the method steps according to the invention, and a virtually quantitative separation of valuable xylose is possible at the same time.

Further advantages of the method according to the invention include a shorter heating-up time and reaction time in prehydrolysis and a low bath ratio of water-to-wood chips in the first method step. Further, steam is used, which has the advantage that the decomposed hemicelluloses occur in a more highly concentrated form than if water prehydrolysis were carried out. The steam condensate then essentially precipitates as prehydrolysate.

Further, a prehydrolysis of the wood chips by the method according to the invention saves considerable time and money compared with conventional methods. In addition, the hydrolysate contains sugars in a high concentration of about 12% to 14%, which likewise provides an advantageous foundation for further processing or, if desired, further concentration. The mechanical pulps contain, e.g., after countercurrent washing, only about 10% or less of the original hemicelluloses, depending on the intensity of the impregnation and on the prehydrolysis conditions.

Accordingly, a virtually complete removal of hemicelluloses from the wood can be achieved without any unwanted mixing of lignin and carbohydrates. Much more, a clear separation of the essential wood constituents in pure form is achieved, i.e., an effective separation of all soluble carbohydrates is achieved in one separating step with, in addition, low cellulose losses.

A continuous operating mode is likewise possible according to the invention. Disposal problems related to waste water disposal, burning and the like are eliminated. Further, additional process steps for working up the hemicelluloses for their disposal can be dispensed with. Additional costs for disposal of waste products can likewise be avoided.

In addition, valuable products in the form of hemicelluloses, particularly xylans, are obtained and, if desired, can be further refined in an appropriate manner. Moreover, commercial exploitation of xylose for further processing to form xylite (xylitol) is very important. This contributes considerably to improving the economic efficiency of the method according to the invention. According to the invention, a prehydrolysate which is free of foreign substances is obtained and high xylose concentrations are achieved in the prehydrolysate. Costly concentration of xylose is generally not necessary and it is also unnecessary to bleach the xylose that is obtained. A separation of lignosulfonates which is generally required in the prior art can likewise be omitted.

Further, formation of furfural is extensively prevented by the method according to the invention, so that there are no losses in output in this respect. Instead, the desired high yields of hemicelluloses, which are broken down into sugars, and high yields of α-cellulose are obtained.

The invention will be explained further by the following examples.

### DESCRIPTION OF THE PREFERRED EMBODIMENT EXAMPLES

### Examples

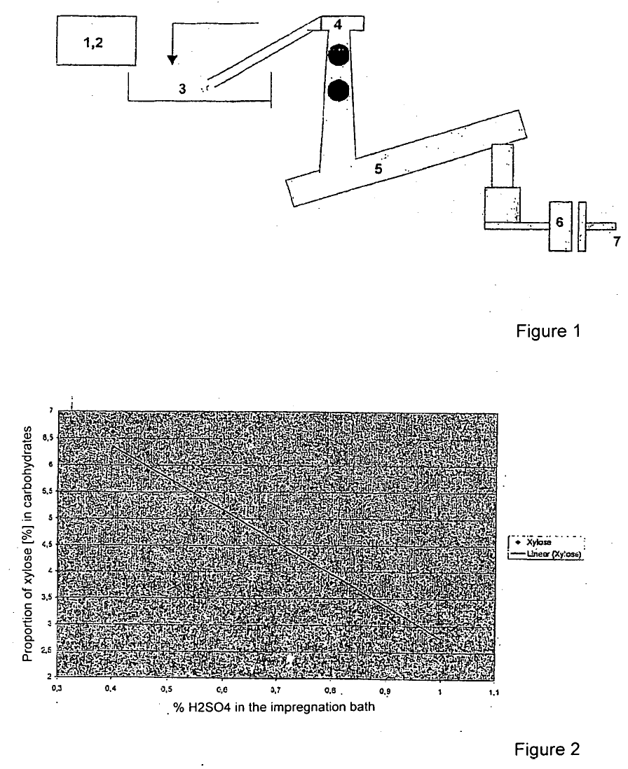

Some embodiment forms of the method according to the invention will be illustrated in detail with reference to the accompanying FIGS. 1 and 2.

As is shown in FIG. 1, uniformly comminuted commercial wood chips (1) from which dirt has been removed are first moved along the MSD (2), wherein the wood structure is extensively destroyed and the natural moisture is considerably reduced (by approximately 50% to approximately 20-25%). By MSD is meant a worm press with the characteristic that the shaft of the worm compresses with increasing distance from the feed point of the wood chips. Accordingly, there is less free space available for the wood chips. The worm rotates slowly and the wood chips are conveyed into a perforated steel plate. The water which exits as a result of the crushing and pressing of the wood chips can be discharged through the perforated steel plate.

As a result of the treatment described above, the wood chips are absorbent for the diluted acid (3) which is added, as indicated by the arrow, and required in the prehydrolysis for decomposing the hemicelluloses contained in the wood. In the present case, a beech wood sample was impregnated with 0.6-percent sulfuric acid and subsequently hydrolyzed at 160° C. for 20 minutes.

The wood chips which are impregnated with acid can be conveyed (4) to the charging hopper of the digester by means of a perforated conveyor belt or a perforated channel with an archimedean screw. Due to the obviously very uniform distribution of the acid, the hydrolysis of the wood chips can be carried out completely in only twenty minutes with steam. The wood chips are conveyed through the conveying screw installed in the digester and emptied with a continuous discharge via a blow valve connected to a cyclone. Alternatively, a refiner or a deknotter can also be provided in order that parts still having the wood chip structure are well digested but also defibrated. By deknotter is meant a machine for comminuting steam-treated wood chips that have not been defibrated. The hydrolysate containing the decomposed hemicelluloses in the form of monosugars must also be drawn off continuously at the other end of the digester. This makes it possible for a production plant to monitor the hydrolysate composition likewise continuously. This measurement signal can be utilized for statistical process control.

A good defibration, for example, in a refiner (6), of the prehydrolyzed wood chips is advantageous for the subsequent washing step (7) in which the sugars still adhering to the fibers are separated quantitatively. A multistep countercurrent wash is advantageous for this purpose. Separation can also be carried out by centrifuging which can be sufficient for quantitative removal of soluble sugars from the prehydrolyzed wood, possibly in combination with one or two washing steps in the centrifuge.

FIG. 2 shows the influence of the sulfuric acid concentration on the xylose content in beech wood. In particular, FIG. 2 shows, on the one hand, that the prehydrolysis step with MSD/impregnation and digester illustrated in FIG. 1 is reproducible and, further, that there is a linear relationship between the sulfuric acid concentration in the impregnating bath and the xylose content in the fibrous material after washing, which can be made use of for optimizing in a corresponding manner.

An analysis of the cellulose yield in a beech wood treated using the method according to the invention followed by five-step washing gives the following results:

TABLE 3

| | % H₂SO₄ during | Cellulose loss | Yield |
|---|---|---|---|
| Sample | impregnation | [%] | [%] |
| V1 | 0.6 | 6.6 | 93.5 |
| V2 | 0.8 | 6.75 | 93.25 |
| V3 | 1.0 | 7.8 | 92.2 |

The cellulose loss—measured as glucose—is probably much lower because glucomannan is also taken into account as a glucose source in the prehydrolysis and is certainly easier to break down than the cellulose chains themselves. Accordingly, the yield is substantially higher than in the prior art methods in which the basic component is sodium hydroxide.

Further, the xylose content was determined in 12.3 kg of a beech wood that is used. The complete composition of the beech wood is given in the additional information in “Paper [Das Papier]” (53(2), (1999) 101:

TABLE 4

| Constituent | [%] |
|---|---|
| Rhamnose | 0.42 |
| Mannose | 1.18 |
| Arabinose | 0.15 |
| Galactose | 0.95 |
| Xylose | 18.70 |
| Glucose | 43.41 |
| 4-O—Me | 1.03 |

The results are shown in the following Table 5:

TABLE 5

| | Solid | Liquid | | Difference |
|---|---|---|---|---|
| | determined | determined in | Total | between |
| Calculated prior | in fibrous | condensate and | solid + | calculated |
| to prehydrolysis | material | wash water | liquid | and actual |
| (28%)*) 3.44 kg | 0.634 kg | 2.69 kg | 3.324 kg | 0.12 kg |

*)12.3 kg beech wood × 0.28; including glucoronic acid

Therefore, it may be said that there is a satisfactory correspondence (about 3.5% deviation) in comparing between the calculated xylose content of the beech and the total xylose contents in the fibrous materials, condensate and wash water. When using a 0.6-percent sulfuric acid in the impregnation, there is a residual content of xylose of about 5% after washing. Accordingly, it was possible to obtain 78% of the xylose contained in the beech wood. The proportion of xylose in the dissolved carbohydrates is between 68% and 75% depending on the test conditions.

This result represents a considerable improvement over the prior art.

While the foregoing description and drawings represent the present invention, it will be obvious to those skilled in the art that various changes may be made therein without departing from the true spirit and scope of the present invention.

1-11. (cancelled).

12. A method for separating xylose from lignocelluloses rich in xylan, particularly wood, and for obtaining pulp, comprising continuous implementation of the following steps:
(1) pretreating wood chips through mechanical destruction of the original structure by crushing and/or chopping the wood chips;
(2) impregnating the obtained wood mass with diluted mineral acid;
(3) carrying out a prehydrolysis of the obtained wood mass modified by the process under the influence of steam at a temperature of at least approximately 140° C. for approximately 5 to 40 minutes to hydrolyze the obtained hemicelluloses; and
(4) removing the hemicelluloses from the residual pulp by filtering and/or centrifuging while obtaining an aqueous solution rich in xylose.

13. A method for separating xylose from lignocelluloses rich in xylan, particularly wood, and for obtaining pulp, comprising continuous implementation of the following steps:
(1) providing comminuted vegetable materials, particularly straw and bagasse;
(2) impregnating the comminuted vegetable materials with diluted mineral acid;
(3) carrying out a prehydrolysis of the comminuted vegetable materials modified by the process under the influence of steam at a temperature of at least approximately 140° C. for approximately 5 to 40 minutes to hydrolyze the obtained hemicelluloses; and
(4) removing the hemicelluloses from the residual pulp by filtering and/or centrifuging while obtaining an aqueous solution rich in xylose.

14. The method according to claim 12, comprising that the pulp obtained by filtering and/or centrifuging is washed.

15. The method according to claim 13, comprising that the pulp obtained by filtering and/or centrifuging is washed.

16. The method according to claim 13, comprising that hardwood is used as wood for the wood chips.

17. The method according to claim 16, wherein the hardwood is beech or eucalytus.

18. The method according to claim 13, wherein a press worm or a spindle press is used for the mechanical destruction according to (1).

19. The method according to claim 12, wherein sulfuric acid, hydrochloric acid and/or nitric acid are/is used as mineral acid.

20. The method according to claim 13, wherein sulfuric acid, hydrochloric acid and/or nitric acid are/is used as mineral acid.

21. The method according to claim 19, wherein an approximately 0.05 N to 2.5 N diluted aqueous mineral acid solution is used.

22. The method according to claim 21, wherein an approximately 0.1 N to 1.5 N diluted aqueous mineral acid solution is used.

23. The method according to claim 20, wherein an approximately 0.05 N to 2.5 N diluted aqueous mineral acid solution is used.

24. The method according to claim 23, wherein an approximately 0.1 N to 1.5 N diluted aqueous mineral acid solution is used.

25. The method according to claim 12, wherein the treatment with steam in step (3) is carried out at a temperature of approximately 140° C. to 180° C., particularly for approximately 5 to 20 minutes.

26. The method according to claim 25, wherein the treatment with steam in step (3) is carried out at a temperature of approximately 150° C. to 180° C.

27. The method according to claim 13, wherein the treatment with steam in step (3) is carried out at a temperature of approximately 140° C. to 180° C., particularly for approximately 5 to 20 minutes.

28. The method according to claim 27, wherein the treatment with steam in step (3) is carried out at a temperature of approximately 150° C. to 180° C.

29. The method according to claim 13, wherein the wood mass modified by the process is further defibrated according to step (3).

30. The method according to claim 29, wherein defibration is carried out with a refiner, a toothed disk mill or via a steam bubble line.

31. The method according to claim 14, wherein washing is carried out with hot water or ammonia water.

32. The method according to claim 31, wherein washing is carried out as a one-step or multiple-step countercurrent wash.
